(19) United States
(12) Patent Application Publication
(10) Pub. No.: US 2004/0147534 A1
(43) Pub. Date: Jul. 29, 2004

(54) Topical composition and method for treating occlusive wounds

(76) Inventors: Mary Ann Foote, Aurora, IL (US); Mark Harlan Mandel, Schaumburg, IL (US)

Correspondence Address: Faier & Faier, P.C., 566 W. Adams Street, # 600, Chicago, IL, 60661, US

(21) Appl. No.: 10/350,200

(22) Filed: Jan. 23, 2003

(51) Int. Cl. A61K 31/522; A61K 31/455

(52) U.S. Cl. 514/263.32; 514/355

(57) ABSTRACT

The present invention provides a topical composition comprising about 6% to about 15% nifedipine and about 6% to about 15% pentoxifylline for treating severe vascular occlusive wounds. The present invention also provides a method and a kit for treating the vascular occlusive wound by applying the composition to the open wound, and cleaning and dressing the wound at least once daily.

### FIELD OF THE INVENTION

The present invention provides a topical composition, a method and a kit for treating vascular occlusive wounds.

### BACKGROUND

Modern technology provides a number of ways to treat open wounds that have access to an adequate supply of blood, which provides oxygen and other healing factors to the wound. However, current technology provides very few methods to treat end-stage vascular wounds that are encumbered by peripheral vascular disease, otherwise known as occlusive wounds. Occlusive wounds are particularly difficult to treat because the absence of vascular circulation prevents normal healing factors from reaching the wound. Unfortunately, an increase in the incidence of diabetes has resulted in an increase in difficult-to-treat occlusive diabetic ulcers. Occlusive wounds also result from cardiac disease, trauma, bums and frostbite that are so severe that the body attempts to isolate the wound from healthy tissue.

Some methods to treat such wounds include re-vascularizing the wound by applying chemical fibrinolytic agents, by physically pulverizing the blockage, by pulverizing the blockage by ultrasound, or by using a balloon to open the vascular channels. Other methods include subjecting the patient to hyperbaric oxygen, providing an arterial assist device, such as a vascular stent, and treatment with topical agents or non-occlusive dressings that do not promote circulation, but may stabilize the wound, debride the wound or create an inflammatory response to help promote healing. However, if the foregoing treatments are not available or are ineffective (as is the case for many of the more severe cases), the prognosis generally indicates excision of the afflicted tissue, including amputation of an afflicted limb.

Efforts to treat external occlusive wounds by topical application of nifedipine have been reported. Attempts to treat occlusive wounds have also included the use of low amounts of pentoxifylline and nifedipine in conjunction with therapeutic agents, such as bacitracin, polymyxin, phenytoin sodium and misoprostol. However, it is known that application of nifedipine to areas having vascular circulation can result in systemic introduction of the nifedipine, leading to hypotension. Therefore, the ordinary artisan would not be expected to increase the concentration of nifedipine as an occlusive wound treatment. Moreover, the state of the art indicates applying therapeutic agents to the periphery of such wounds. Therefore, the ordinary artisan would not be expected to apply a therapeutic agent directly to the open wound. In addition, the ordinary artisan would not know to cover an occlusive wound with a saline soaked gauze, after application of a topical occlusive wound treatment, since the soaked gauze may interfere with the action of the topical treatment.

### SUMMARY OF THE INVENTION

The present invention provides a topical composition comprising about 6% to about 15% nifedipine and about 6% to about 15% pentoxifylline for treating severe vascular occlusive wounds. The present invention also provides a method and a kit for treating the vascular occlusive wound by applying the composition to the open wound, and cleaning and dressing the wound at least once daily.

### DETAILED DESCRIPTION

The invention is described by the following examples. It should be recognized that variations based on the inventive features disclosed herein are within the skill of the ordinary artisan, and that the scope of the invention should not be limited by the examples. To properly determine the scope of the invention, an interested party should consider the claims herein, and any equivalent thereof. In addition, all citations herein are incorporated by reference.

The present invention provides a topical composition comprising nifedipine and pentoxifylline for treating severe wounds, a method and a kit for treating the severe wound by repeatedly applying the composition to the wound, and cleaning the wound. Nifedipine is a known calcium channel antagonist. Pentoxifylline is a known phosphodiesterase inhibitor. In the context of the present invention, the terms nifedipine and pentoxifylline include all therapeutically acceptable forms of the compounds, such as salts, hydrates and derivatives thereof. Some examples of occlusive wounds include diabetic micro-occlusion and combination venous and/or arterial wounds.

The topical composition according to the invention comprises about 6% to about 15% nifedipine and about 6% to about 15% pentoxifylline. In an embodiment, the topical composition contains about 8% to about 13% nifedipine and about 6% to about 12% pentoxifylline. In another embodiment, the topical composition according to the invention contains about 8% to about 11% nifedipine and about 9% to about 12% pentoxifylline. In a further embodiment, the topical composition according to the invention contains about 10% nifedipine and about 10% pentoxifylline. In yet another embodiment, the topical composition consists essentially of a therapeutically effective amount of nifedipine, a therapeutically effective amount of pentoxifylline, and optional therapeutic agents in a carrier.

Examples of optional therapeutic agents includes antibiotics, potentiating agents, pain treatment agents and mixtures thereof. In an embodiment, the composition contains a therapeutic agent selected from the group consisting of morphine (preferably about 1%), clonidine, baclofen, gabapentin, gentamicin, bacitracin, polymyxin, phenytoin sodium, misoprostol, ibuprofen (preferably about 2%), metronidazole and a mixture thereof. In a further embodiment, the composition contains morphine, ibuprofen, or a mixture of bacitracin, polymyxin, phenytoin sodium and misoprostol.

The topical composition is applied externally as a cream, a gel, an ointment or a lotion. In an embodiment, the carrier is a cream. In one embodiment, the topical composition includes an Enhanced Absorption Vehicle (EAV) in the carrier or as the carrier. In another embodiment, the EAV comprises cetyl alcohol, stearyl alcohol, stearic acid, glyceryl monostearate, isopropyl myristate, lecithin/isopropyl palmitate solution, urea and purified water. In a further embodiment the EAV further contains BHT, simethicone, potassium sorbate, sodium hydroxide, polyoxyethylene and EDTA. Moreover, the EAV may also contain sodium lauryl sulfate, glycerin, sorbitol and lactic acid. The foregoing formulation is also known as a vanishing enhanced penetration cream. One acceptable vehicle is VanPen a carrier that is commercially available from the Professional Compounding Centers of America (PCCA). The VanPen may be further modified by the addition of ibuprofen as a pain reliever and potentiating agent, gelling agent/emulsifier (such as, for example, one of the Tween or Span emulsifiers, Tween 80 or Span 80), and/or squalene as a moisturizing agent.

In another embodiment, the carrier is selected from the group consisting of hydroxypropyl cellulose (i.e., 2% aqueous HPC), hydroxyethyl cellulose (4% aqueous HEC), aquaphor, simple vanishing cream and a mixture thereof. In a further embodiment the carrier is, or contains, an EAV selected from the group consisting of pluronic organogel (PLO 20%) or a squalene containing penetrating cream (e.g. Lipoderm™ from PCCA), and a mixture thereof.

Without being limited by any particular theory, it is believed that the topical composition according to the invention improves vasodilation, capillary flow, granulation and epithelialization.

Exemplary Case Studies

Patients with severe, known peripheral vascular disease that were not candidates for re-vascularization, and non-responsive to current wound management methods, such as hydrogels, hydrocolloids, regranulating agents such as, for example, that sold as Regranex® and debriding agents were treated with a topical composition according to the invention. In all cases, the wounds all suffered vascular or micro-occlusion, with an absence of or significantly reduced blood flow to the wound. In many cases, many patients also suffered poor nutritional intake, which exacerbated the severity of their wounds.

On the average, the typical wound in the case studies had an area of about 13 cm by 3 cm, and a core depth of about 0.7 cm. As expected, doppler studies of the patents exhibited little to no blood flow to the extremities. In addition, the ankle-brachial index (ABI) ratios were so small as to be unobtainable or were much less than 0.6.

The protocol for treating the patients varied according to the severity of the wound. In general, the wound is cleaned with a spray bottle of isotonic saline solution. A thin layer of the topical composition is applied to the open wound with a sterile applicator. The affected area is covered with a isotonic saline soaked sterile gauze, and loosely wrapped with a breathable gauze such as, for example, gauze wrap (e.g. Kerlix™ from Kendall). The procedure is performed at least once daily, preferably twice daily. In lieu of, and/or in addition to covering the wound with the soaked sterile gauze that is loosely wrapped, hyperbaric oxygen may be used to treat the wound in conjunction with the application of the topical composition.

Wound healing in the form of wound granulation and-dimension contraction was observed as quickly as 10 days after the start of treatment. In some cases the healing time was decreased by as much as 70%. In addition, the amount of devitalized tissued decreased without the use of any other topical agent, and exposed tendons were covered with granulating tissue.

Moreover, no significant systemic absorption was observed for the vascular occluded patient. Systemic absorption presents the potential for harmful side-effects and/or drug-drug interactions that could interfere with a patient's oral regime. Without being limited or bound by any particular theory, it is believed that the non-viable tissue acts as a self-limiting barrier to decrease the potential for interaction between the topical composition and any systemic therapeutics. In addition, minor spillage to the periphery of the wound was not found to adversely affect the patient. The non-viable tissue may also localize the effective agents, and increase local duration of effect of the effective ingredients by retarding the metabolism of the active ingredients.

As local tissue epithelialization and possible microvascular neogenesis occur, there may be increased pain awareness. However, the increased local discomfort is treated by adding an anesthetic agent to the topical composition to relieve the pain. In the context of the ensuing cases which are representative and not comprehensive, all percentages are weight percent.

Case 1

Patient one was a female (age 94) that suffered from peripheral vascular disease, hypertension, congestive heart failure, obesity and immobility. Her wound was non-responsive to conventional treatment for at least 18 months prior to the treatment discussed herein. Her wound covered the entire anterior and lateral surface of her foot with the tendons fully exposed. The wound measured 13.4 cm by 5.5 cm, with a core depth of 0.7 cm at the dorsum and 100% slough of devitalized tissue. The wound measured 9 cm by 7 cm, with a core depth of 0.8 cm at the ankle. A composition comprising 8% nifedipine and 10% pentoxifylline was applied to the wound 2 times daily for about a month. The wound contraction was apparent as of 14 days. Significant improvement was observed at six weeks with decreased slough, granulation over tendons and increased sensory awareness.

Case 2

Patient two was a female (age 96) that suffered from peripheral vascular disease, dementia, osteomyelitis, immobility and gangrene of the foot. Her wound covered the right gaiter region of the leg and was treated by conventional means for at least eight months prior to the treatment discussed herein. Consulted physicians recommended amputation. The wound measured 13.6 cm by 3.5 cm, with a core depth of 0.3 cm and a 70% slough of devitalized tissue. A composition comprising 8-10% nifedipine and 10% pentoxifylline was applied to the wound 2 times a day for about 3½ months. The wound contraction was apparent as of 14 days. Significant improvement with progressive granulation development was observed with the gaiter wound granulated to closure. The gangrene was treated by conventional means (e.g. systemic and topical antibiotics), without significant observed interaction with the topical treatment.

Case 3

Patient three was a female (age 88) that suffered from peripheral vascular disease, congestive heart failure, arterial sclerotic heart disease and cerebral vascular accident. Her wound covered the right gaiter and was present for at least 9 months (7 months of hydrocolloid treatment) prior to the treatment discussed herein. The wound measured 14.7 cm by 2.1 cm, with a core depth of 1 cm and 60% slough of devitalized tissue. A composition comprising 8-10% nifedipine and 10% pentoxifylline was applied to the wound 2 times a day for about 3 months. The wound contraction was apparent as of 14 days. Significant improvement with progressive granulation development was observed with the healing of the wound.

We claim:

1. A method of treating an occlusive wound comprising:
cleaning the wound with an isotonic cleanser;
applying to the open wound, a topical composition comprising a carrier, nifedipine, pentoxifylline and an optional therapeutic agent; and
performing the procedure at least once daily.

2. The method according to claim 1, further comprising the step of covering the wound with saline moistened gauze after application of the topical composition.

3. The method according to claim 1, further comprising the step of treating the wound with hyperbaric oxygen after application of the topical composition.

4. The method according to claim 1, further comprising the steps of covering the wound with saline moistened gauze after application of the topical composition, and treating the wound with hyperbaric oxygen.

5. The method according to claim 1, wherein the wound is cleaned and treated twice daily.

6. The method according to claim 1, wherein the carrier is a cream comprising cetyl alcohol, stearyl alcohol, stearic acid, glyceryl monostearate, isopropyl myristate, lecithin/isopropyl palmitate solution, urea, BHT, simethicone, potassium sorbate, sodium hydroxide, polyoxyethylene, EDTA and purified water, and the wound is cleaned and treated twice daily.

7. The method according to claim 6, wherein the carrier further comprises an ingredient selected from the group consisting of ibuprofen, gelling agent, squalene and a mixture thereof.

8. The method according to claim 1, wherein the carrier is selected from the group consisting of aqueous hydroxypropyl cellulose, hydroxyethyl cellulose, pluronic organogel, vanishing cream, aquaphor, squalene containing penetrating cream and a mixture thereof.

9. The method according to claim 1, wherein the topical composition contains about 6% to 15% nifedipine and about 6% to 15% pentoxifylline.

10. The method according to claim 1, wherein the topical composition contains about 8% to 13% nifedipine and about 6% to 12% pentoxifylline.

11. The method according to claim 1, wherein the topical composition contains about 8% to 11% nifedipine and about 9% to 12% pentoxifylline.

12. The method according to claim 1, wherein the topical composition contains about 10% nifedipine and about 10% pentoxifylline.

13. The method according to claim 1, wherein the topical composition contains the optional therapeutic agent.

14. The method according to claim 13, wherein the therapeutic agent is selected from the group consisting of morphine, clonidine, baclofen, gabapentin, gentamicin, bacitracin, polymyxin, phenytoin sodium, misoprostol, ibuprofen, metronidazole and a mixture thereof.

15. The method according to claim 1, further comprising the step of covering the wound with saline moistened gauze after application of the topical composition, wherein the wound is cleaned and treated twice daily, the carrier is a cream comprising cetyl alcohol, stearyl alcohol, stearic acid, glyceryl monostearate, isopropyl myristate, lecithin/isopropyl palmitate solution, urea, BHT, simethicone, potassium sorbate, sodium hydroxide, polyoxyethylene, EDTA, ibuprofen, gelling agent, squalene and purified water, and the topical composition contains about 10% nifedipine and about 10% pentoxifylline.

16. A topical composition consisting essentially of a carrier, nifedipine and pentoxifylline, wherein the nifedipine and the pentoxifylline are present in therapeutically effective concentrations for the treatment of occlusive wounds.

17. The topical composition according to claim 16, wherein the carrier is a cream comprising cetyl alcohol, stearyl alcohol, stearic acid, glyceryl monostearate, isopropyl myristate, lecithin/isopropyl palmitate solution, urea, BHT, simethicone, potassium sorbate, sodium hydroxide, polyoxyethylene, EDTA and purified water.

18. The topical composition according to claim 17, wherein the carrier further comprises an ingredient selected from the group consisting of ibuprofen, gelling agent, squalene and a mixture thereof.

19. The topical composition according to claim 16, wherein the carrier is selected from the group consisting of aqueous hydroxypropyl cellulose, hydroxyethyl cellulose, pluronic organogel, vanishing cream, aquaphor, squalene containing penetrating cream and a mixture thereof.

20. The topical composition according to claim 16, having about 6% to 15% nifedipine and about 6% to 15% pentoxifylline.

21. The topical composition according to claim 16, having about 8-11% nifedipine and about 9-12% pentoxifylline.

22. The topical composition according to claim 21 containing an optional therapeutic agent.

23. The topical composition according to claim 20, containing an therapeutic agent selected from the group consisting of morphine, clonidine, baclofen, gabapentin, gentamicin, bacitracin, polymyxin, phenytoin sodium, misoprostol, ibuprofen, metronidazole and a mixture thereof.

24. A topical composition comprising a carrier, about 6% to 15% nifedipine, about 6% to 15% pentoxifylline and an optional therapeutic agent.

25. The topical composition according to claim 24, wherein the carrier is a cream comprising cetyl alcohol, stearyl alcohol, stearic acid, glyceryl monostearate, isopropyl myristate, lecithin/isopropyl palmitate solution, urea, BHT, simethicone, potassium sorbate, sodium hydroxide, polyoxyethylene, EDTA and purified water.

26. The topical composition according to claim 25, wherein the carrier further comprises an ingredient selected from the group consisting of ibuprofen, gelling agent, squalene and a mixture thereof.

27. The topical composition according to claim 24, wherein the carrier is selected from the group consisting of aqueous hydroxypropyl cellulose, hydroxyethyl cellulose, pluronic organogel, vanishing cream, aquaphor, squalene containing penetrating cream and a mixture thereof.

28. The topical composition according to claim 24, having about 8% to 13% nifedipine and about 6% to 12% pentoxifylline.

29. The topical composition according to claim 24, having about 8% to 11% nifedipine and about 9% to 12% pentoxifylline.

30. The topical composition according to claim 24, having about 10% nifedipine and about 10% pentoxifylline.

31. The topical composition according to claim 24 containing the optional therapeutic agent.

32. The topical composition according to claim 31, wherein the therapeutic agent is selected from the group consisting of morphine, clonidine, baclofen, gabapentin, gentamicin, bacitracin, polymyxin, phenytoin sodium, misoprostol, ibuprofen, metronidazole and a mixture thereof.

33. A kit for treating an occlusive wound comprising:
a topical composition comprising a carrier, nifedipine, pentoxifylline and an optional therapeutic agent, wherein the nifedipine and the pentoxifylline are present in therapeutically effective concentrations for the treatment of occlusive wounds; and
instructions directing a treatment provider to clean the wound with an isotonic cleanser;
to apply the topical composition to the open wound; and
to perform the procedure at least once daily.

34. The kit according to claim 33, wherein the instructions further direct the treatment provider to cover the wound with saline moistened gauze after application of the topical composition.

35. The kit according to claim 33, wherein the direction further instruct the treatment provider to treat the wound with hyperbaric oxygen after application of the topical composition.

36. The kit according to claim 33, wherein the direction further instruct the treatment provider to cover the wound with saline moistened gauze after application of the topical composition and treat the wound with hyperbaric oxygen.

37. The kit according to claim 33, wherein the carrier comprises cetyl alcohol, stearyl alcohol, stearic acid, glyceryl monostearate, isopropyl myristate, lecithin/isopropyl palmitate solution, urea, BHT, simethicone, potassium sorbate, sodium hydroxide, polyoxyethylene, EDTA and purified water, and the instructions direct the treatment provider to clean and treat the wound twice daily.

38. The kit according to claim 37, wherein the carrier further comprises an ingredient selected from the group consisting of ibuprofen, gelling agent, squalene and a mixture thereof.

39. The kit according to claim 33, wherein the carrier is selected from the group consisting of aqueous hydroxypropyl cellulose, hydroxyethyl cellulose, pluronic organogel, vanishing cream, aquaphor, squalene containing penetrating cream and a mixture thereof.

40. The kit according to claim 33, wherein the topical composition comprises about 6% to 15% nifedipine and about 6% to 15% pentoxifylline.

41 The kit according to claim 33, wherein the topical composition comprises about 8% to 13% nifedipine and about 6% to 12% pentoxifylline.

42 The kit according to claim 33, wherein the topical composition comprises about 8% to 11% nifedipine and about 9% to 12% pentoxifylline.

43. The kit according to claim 33, wherein the topical composition comprises about 10% nifedipine and about 10% pentoxifylline.

44. The kit according to claim 33, wherein the instructions direct the treatment provider to clean and treat the wound twice daily.

45. The kit according to claim 33, wherein the topical composition contains the optional therapeutic agent.

46. The kit according to claim 45, wherein the therapeutic agent is selected from the group consisting of morphine, clonidine, baclofen, gabapentin, gentamicin, bacitracin, polymyxin, phenytoin sodium, misoprostol, ibuprofen, metronidazole and a mixture thereof.

47. The kit according to claim 33, wherein the topical composition consists essentially of a carrier, nifedipine and pentoxifylline.

48. The kit according to claim 33, wherein the instructions further direct the treatment provider to cover the wound with saline moistened gauze after application of the topical composition, the carrier comprises cetyl alcohol, stearyl alcohol, stearic acid, glyceryl monostearate, isopropyl myristate, lecithin/isopropyl palmitate solution, urea, BHT, simethicone, potassium sorbate, sodium hydroxide, polyoxyethylene, EDTA, ibuprofen, gelling agent, squalene and purified water, the instructions direct the treatment provider to clean and treat the wound twice daily, and the topical composition comprises about 10% nifedipine and about 10% pentoxifylline.
